(19) United States
(12) Patent Application Publication
(10) Pub. No.: US 2023/0395242 A1
(43) Pub. Date: Dec. 7, 2023

(54) SYSTEMS AND METHODS OF TRACKING DENTAL PATIENT LIFE CYCLES AND ROI ON MARKETING EFFORTS

(71) Applicant: Ryan Hemphill, Rosevill, CA (US)

(72) Inventor: Ryan Hemphill, Rosevill, CA (US)

(21) Appl. No.: 18/130,243

(22) Filed: Apr. 3, 2023

Related U.S. Application Data
(60) Provisional application No. 63/326,509, filed on Apr. 1, 2022.

(51) Int. Cl. G16H 40/20; G06Q 30/0201

(52) U.S. Cl. CPC G16H 40/20; G06Q 30/0201

(57) ABSTRACT

A system for providing a patient management platform for a dental practice is disclosed, including at least one user computing device in operable connection with a user network. An application server is in operable communication with the user network to host an application program for providing a patient management platform. The application program includes a user interface module for providing access to the application program via the at least one user computing device. An ROI calculation module calculates an ROI for a dental practice using a plurality of patient profile information and a plurality of marketing information. Matching logic generates operation metric calculations.

### CROSS-REFERENCE TO RELATED APPLICATIONS

The present application claims priority to U.S. Provisional Application No. 63/326,509 filed Apr. 1, 2022, titled “SYSTEMS AND METHODS OF TRACKING DENTAL PATIENT LIFE CYCLES AND ROI ON MARKETING EFFORTS” which is hereby incorporated by reference in its entirety.

### TECHNICAL FIELD

Embodiments of the invention relate to systems and methods for tracking patient life cycles for new dental patients and real-time return on investment (ROI) of marketing efforts for dental patients.

### BACKGROUND

Dental practices are among traditional professional businesses wherein existing technology fails to segment and then match marketing attribution data to patient data. This often results in severing the link between patient data and marketing data. Unfortunately, this can reduce patient retention efforts, service recommendations, profitability, and overall knowledge of the health of the dental practice and its operational successes and failures. In particular, the lack of a link between patient data and marketing data means that there is little, if any, predictability for growing the dental practice using marketing activities because positive indicators of past performance are not tracked. Other traditional professions may also suffer from similar issues, including those in the health care provider and/or medical industries, legal industry, accounting, and others.

In general, the only data that is created and stored in the dental industry is often data that is harvested and/or stored after being manually inputted into the practice management system (PMS). As is common practice in the industry, the data that marketing companies hired by a dental practice can provide also stops being tracked after the potential patient calls and/or reaches out the office. These two data issues are not and cannot be married and matched together currently, due to a lack of integration.

To elaborate, some existing dental industry platforms allow for monitoring call conversion data, production data (by year, month, day, and/or patient), new patient acquisition data, and digital presence data. They may provide generalized marketing/awareness campaigns or advertising, information for patients to research on a practice's website or social media, referrals and surveys, and newsletters and other communications. They may also include call tracking and recording, scoring and analytics, website tracking and conversion pages, brochures, and other information. As stated above, this information is separated from patient visit and/or medical information that could provide valuable insight to a practice and strengthen the practice's ability to serve and retain its patients.

Thus, it would be beneficial to be able to track patient data information and marketing information in an integrated platform, particularly in order to gain valuable insights regarding both.

### SUMMARY OF THE INVENTION

This summary is provided to introduce a variety of concepts in a simplified form that is disclosed further in the detailed description of the embodiments. This summary is not intended for determining or limiting the scope of the claimed subject matter.

The example embodiments provided herein relate to and disclose digital platforms that integrate patient information and marketing information.

In general, the systems and methods disclosed herein are operable to enrich incoming data that is gleaned from marketing initiatives. This data can be injected into one or more data processing pipelines where it is matched with raw data from a dental practice management system's stored data, including patient data. The system can then apply stored logic to the output of this process to determine key performance indicators (KPIs) for a given practice and/or physical office location. Results can include one or more visual representations of actionable data with recommendations for current and future courses of action on a patient-by-patient and/or larger scale basis. Among other things, this can allow the application platform to segment marketing and non-marketing patients and provide a level of clarity and real-time metrics, including marketing return on investment (ROI) information.

The current embodiments permit attribution data enrichment as empowered through matching systems and methods employing logic.

Websites, mobile and tablet applications, and desktop portals are contemplated.

The systems and methods herein are operable to capture each, and every potential interaction point between a patient and the business, including through digital networks, phone calls, interactions with staff, and others.

Other objects and advantages of the various embodiments of the present invention will become obvious to the reader and it is intended that these objects and advantages are within the scope of the present invention. To the accomplishment of the above and related objects, this invention may be embodied in the form illustrated in the accompanying drawings, attention being called to the fact, however, that the drawings are illustrative only, and that changes may be made in the specific construction illustrated and described within the scope of this application.

### BRIEF DESCRIPTION OF THE DRAWINGS

A more complete understanding of the embodiments, and the attendant advantages and features thereof, will be more readily understood by references to the following detailed description when considered in conjunction with the accompanying drawings wherein:

FIG. 1 illustrates a system architecture diagram, according to some embodiments;

FIG. 2A illustrates a system functionality flowchart, according to some embodiments;

FIG. 2B illustrates a system functionality flowchart, according to some embodiments; and

FIG. 3 illustrates an application program in communication with the computer system, according to some embodiments.

### DETAILED DESCRIPTION

The specific details of the single embodiment or variety of embodiments described herein are set forth in this application. Any specific details of the embodiments described herein are used for demonstration purposes only, and no unnecessary limitation(s) or inference(s) are to be understood or imputed therefrom.

Before describing in detail exemplary embodiments, it is noted that the embodiments reside primarily in combinations of components related to particular devices and systems. Accordingly, the device components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

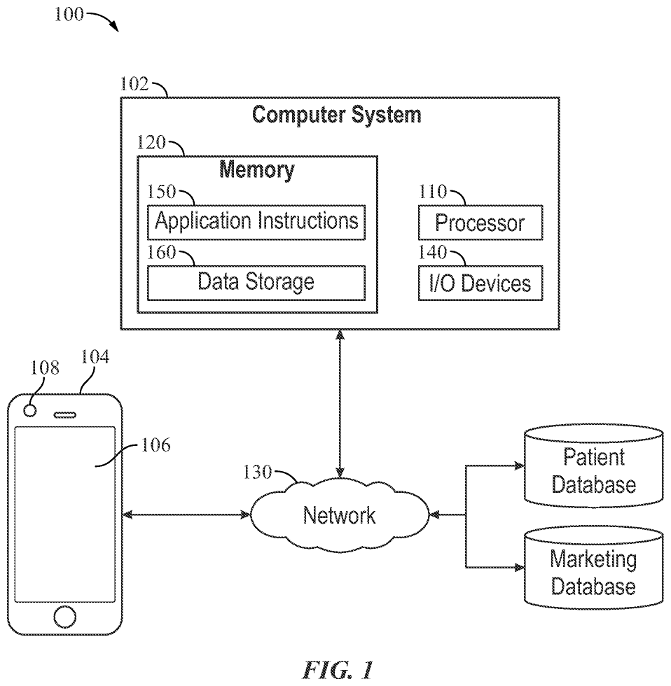

FIG. 1 illustrates a system architecture diagram 100, including a computer system 102, which can be utilized to provide and/or execute the processes described herein in various embodiments. The computer system 102 can be comprised of a standalone computer or mobile computing device, a mainframe computer system, a workstation, a network computer, a desktop computer, a laptop, a tablet, a smartphone, a videogame console, or the like. The computer system 102 includes one or more processors 110 coupled to a memory 120 via an input/output (I/O) interface. Computer system 102 may further include a network interface to communicate with the network 130. One or more input/output (I/O) devices 140, such as video device(s) (e.g., a camera), audio device(s), and display(s) are in operable communication with the computer system 102. In some embodiments, similar I/O devices 140 may be separate from computer system 102 and may interact with one or more nodes of the computer system 102 through a wired or wireless connection, such as over a network interface. In many embodiments, computer system 102 can be a server that is fully automated or partially automated and may operate with minimal or no interaction or human input during processes described herein. As such, many embodiments of the processes described herein can be fully automated or partially automated.

Processors 110 suitable for the execution of a computer program include both general and special purpose microprocessors and any one or more processors of any digital computing device. The processor 110 will receive instructions and data from a read-only memory or a random-access memory or both. The essential elements of a computing device are a processor for performing actions in accordance with instructions and one or more memory devices for storing instructions and data. Generally, a computing device will also include, or be operatively coupled to receive data from or transfer data to, or both, one or more mass storage devices for storing data, e.g., magnetic, magneto-optical disks, or optical disks; however, a computing device need not have such devices. Moreover, a computing device can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive).

A network interface may be configured to allow data to be exchanged between the computer system 102 and other devices attached to a network 130, such as other computer systems, or between nodes of the computer system 102. In various embodiments, the network interface may support communication via wired or wireless general data networks, such as any suitable type of Ethernet network, for example, via telecommunications/telephony networks such as analog voice networks or digital fiber communications networks, via storage area networks such as Fiber Channel storage area networks (SANs), or via any other suitable type of network and/or protocol.

The memory 120 may include application instructions 150, configured to implement certain embodiments described herein, and at least one database or data storage 160, comprising various data accessible by the application instructions 150. In at least one embodiment, the application instructions 150 may include software elements corresponding to one or more of the various embodiments described herein. For example, application instructions 150 may be implemented in various embodiments using any desired programming language, scripting language, or combination of programming languages and/or scripting languages (e.g., C, C++, C #, JAVA®, JAVASCRIPT®, PERL®, etc.).

The steps and actions of the computer system 102 described in connection with the embodiments disclosed herein may be embodied directly in hardware, in a software module executed by a processor, or in a combination of the two. A software module may reside in random-access memory (RAM), flash memory, read-only memory (ROM) memory, erasable programmable read-only memory (EPROM) memory, electrically erasable programmable read-only memory (EEPROM) memory, registers, a hard disk, a solid-state drive (SSD), hybrid drive, dual-drive, a removable disk, a compact disc read-only memory (CD-ROM), digital versatile disc (DVD), high definition digital versatile disc (HD DVD), or any other form of non-transitory storage medium known in the art or later developed. An exemplary storage medium may be coupled to the processor 110 such that the processor 110 can read information from, and write information to, the storage medium. In the alternative, the storage medium may be integrated into the processor 110. Further, in some embodiments, the processor 110 and the storage medium may reside in an Application Specific Integrated Circuit (ASIC). In the alternative, the processor and the storage medium may reside as discrete components in a computing device. Additionally, in some embodiments, the events or actions of a method or algorithm may reside as one or any combination or set of codes and instructions on a machine-readable medium or computer-readable medium, which may be incorporated into a computer program product.

Also, any connection may be associated with a computer-readable medium. For example, if the software is transmitted from a website, server, or other remote source using a coaxial cable, fiber optic cable, twisted pair, digital subscriber line (DSL), or wireless technologies such as infrared, radio, Bluetooth, Wi-Fi, microwave, or others, then the coaxial cable, fiber optic cable, twisted pair, DSL, or wireless technologies such as infrared, radio, Bluetooth, Wi-Fi, microwave, or others can be included in the definition of medium. “Disk” and “disc,” as used herein, include compact disc (CD), laser disc, optical disc, digital versatile disc (DVD), floppy disk, and Blu-ray disc or others where disks usually reproduce data magnetically, while discs usually reproduce data optically with lasers. Combinations of the above should also be included within the scope of computer-readable media.

It should be understood by those in the art that computer system 102 also includes power components that are operably coupled such that the system is operable. This can include one or more batteries if computer system 102 is mobile.

In some embodiments, the system is world-wide-web (www) based, and the network server is a web server delivering HTML, XML, etc., web pages to the computing devices. In other embodiments, a client-server architecture may be implemented, in which a network server executes enterprise and custom software, exchanging data with custom client applications running on the computing device 102.

As shown in the example embodiment, a mobile computing device 104 can also be communicatively coupled with and exchange data with network 130. Those in the art will understand that mobile computing device 104 can include some or all of the same or similar components as computer system 102, coupled to constitute an operable device. Mobile computing device 104 can be a personal digital assistant (PDA), smartphone, tablet computer, laptop, wearable computing device such as a smartwatch or smart glasses, or other device that includes one or more user interface 106, such as a touchscreen and/or audio input/output and/or other display and user input components. Mobile computing device 104 can also include one or more image capturing or reading component 108 (e.g., a digital camera, scanner, or others) and associated structures and elements operatively coupled to at least one processor and memory of the mobile computing device.

Also shown in FIG. 1 are one or more patient database(s) and marketing database(s). These databases can be locally stored in memory or remotely stored in memory that is accessible by computer system 102 via network 130 and may be proprietary, public, or some combination thereof. These databases can also be third-party or system databases in some embodiments and may have one of any manner of structures, privacy measures, and other features and elements. Marketing database(s) can include marketing information that the system 102 stores, uses, makes accessible, and/or sends to users via the network 130. Patient database(s) can include profiles for users and/or patients that includes saved payment information, statistical data, preferences, stored medical data, referrals, and other information for use by computer system 102.

In some embodiments, a variety of features can be provided, and information can be acquired, stored, manipulated, and/or otherwise used to help the practice acquire and retain patients, market, and improve service. Such features and information can include lead data acquisition, API integration, flat file imports, lead enrichment, source information, name information, contact information, associated staff member information, conversion state information, application database storage, dental practice location associated and related information, warehousing for data, PMS integration, API accessing and storing patient and appointment data, patient data, appointment information, transaction information, patient details, treatment plan information, and others. Some or all of these features, data, and information can then be matched and/or otherwise utilized with enriched lead processing to output data sets that are visualized in useful graphs, tables, charts, and other graphics and displays. This can be a form of data hydration, i.e., taking data from a database and injecting it into a dashboard or table or other form of data visualization.

Data enrichment can include taking data and adding and/or applying qualitative metrics. For example, incoming call information can be tagged with data such as “scheduled,” “not scheduled,” “insurance issues,” or a wide variety of other tags, as appropriate. In some embodiments this can be performed manually, while in some embodiments it can be performed automatically, such as through the use of an automated telephone system tracking and storing patient speech or entries on a keypad.

As described herein, data injection can be the movement of data from one location to another. This is most often performed when storing data in databases or moving it into a processing pipeline to have functions performed on it.

Data scraping can include automated processes that are operable to gather information or data from a location that may not be formatted or standardized in the manner that the system can usefully employ. As such, scraping can gather raw data which may then undergo processing to identify or change the data into system usable information.

Matching logic can be employed in order to match particular marketing lead data, offers, and/or campaigns with particular patients who have been the subject of or otherwise interacted with marketing materials. The matching logic can take lead parameters (e.g. name, phone number, email, requested appointment, or others) and match them with patient data within a timeframe that is logical and matching parameters within a certain confidence level. This logic may evolve over time, as some logic is more useful in certain contexts than others. In order to increase a confidence interval over time, data can be run through one or more simulations that can run hypothetical scenarios to output predictive results.

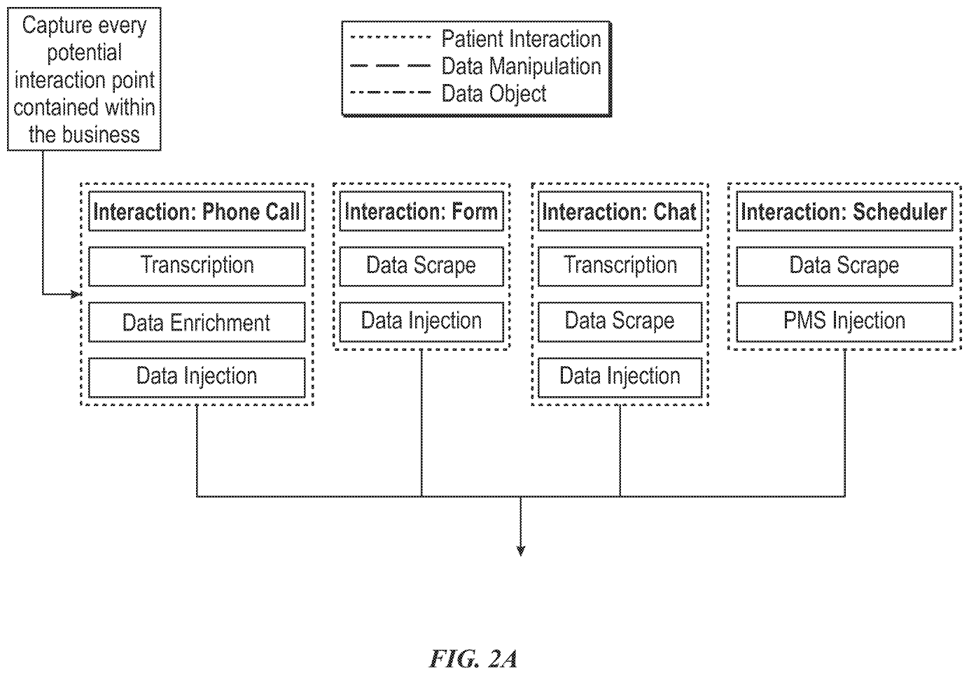

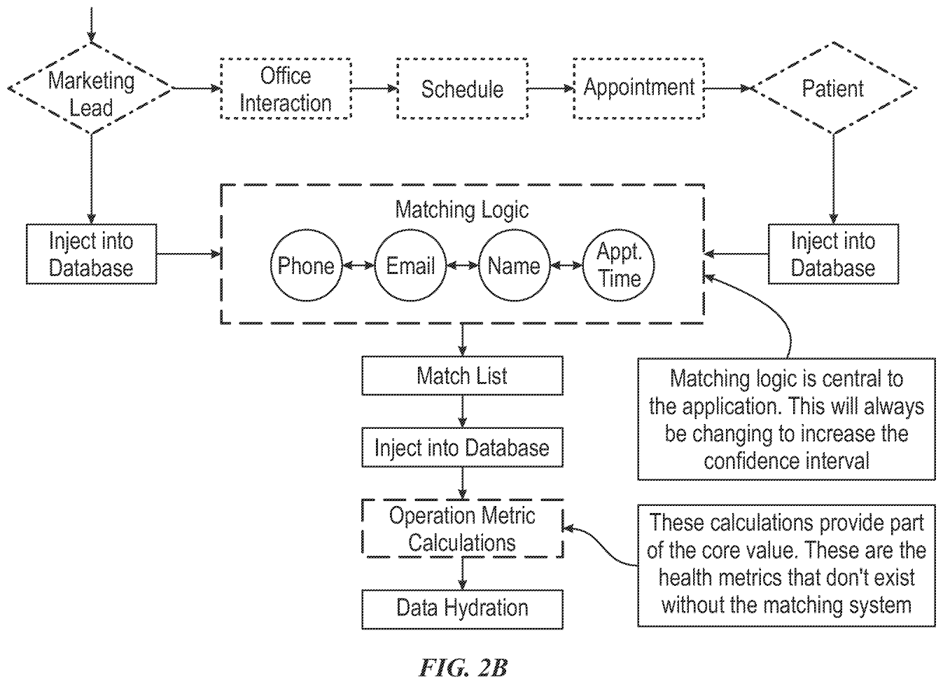

FIGS. 2A-2B show an example embodiment flowchart of an application workflow. As shown, patient interactions, data manipulations, and data objects can be categories used to understand the workflow. Patient interactions can include phone calls, forms, chats, schedulers, office interactions, scheduling, and appointments, among others. Data manipulation can include matching logic and operational metric calculations, among others. Data objects can include marketing leads, appointment data, and patient data.

Operational metrics can include metrics that are used to determine aspects of how patients are interacting with the business. These can include average time to appointment, no show information, and recalls. Average time to appointment may include calculating the average number of business days that a particular office location is open and determining the difference or delta between the date/time for a marketing lead to contact the office and the date/time when the marketing lead actually arrives at the office for an appointment. No show information can be determined by calculating the date/time when an appointment is scheduled and then after a patient or marketing lead was scheduled to show up, using PMS data to determine if the appointment occurred as scheduled or if the patient or marketing lead failed to show up. Recalls can be determined by searching for appointments that are scheduled in the future that have specific codes (e.g., dental codes) that denote that a particular patient or marketing lead is returning in the future for a hygiene or other appointment.

Phone call interactions can include transcription, data enrichment, data injection, and/or other operations. Form interactions can include data scrape, data injection, and other operations. Chat interactions can include transcription, data scrape, data injection, and other operations. Scheduler interactions can include data scrape, PMS injection, and other operations.

Each of phone call interactions, form interactions, chat interactions, and scheduler interactions data can be used to create or otherwise be assigned as marketing lead data object(s). These marketing leads can then lead to office interactions, which may in turn lead to scheduling interactions, which in turn can then lead to appointment interactions, which can each or all be used to create one or more patient data objects. This patient data can be injected into one or more databases (e.g., a patient database). Similarly, marketing lead data objects can be injected into one or more databases (e.g., a marketing database). Information from these databases can then be subjected to matching logic which may include phone, email, name, appointment time, and/or other matching logic functions. Matching logic can be a central focus and feature of the system. This matching logic may be static in some instances, but in most instances, it will be frequently or constantly evolving in order to increase confidence intervals related to the data.

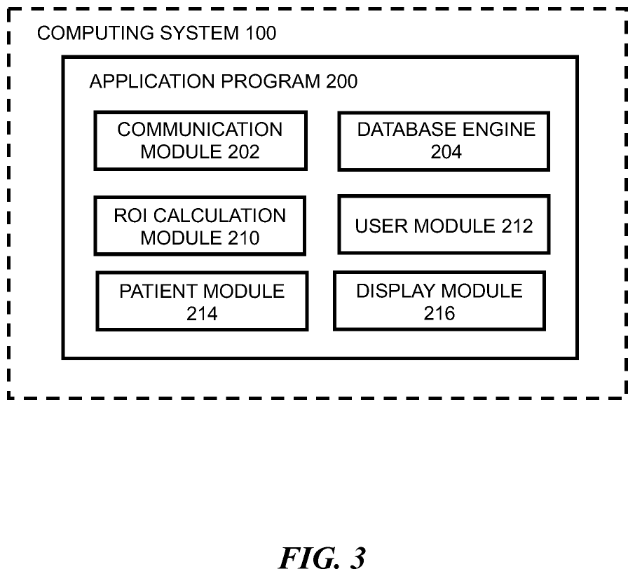

FIG. 3 illustrates an example computer architecture for the application program 200 operated via the computing system 102. The computer system 102 comprises several modules and engines configured to execute the functionalities of the application program 200, and a database engine 204 configured to facilitate how data is stored and managed in one or more databases. In particular, FIG. 3 is a block diagram showing the modules and engines needed to perform specific tasks within the application program 200.

Referring to FIG. 3, the computing system 102 operating the application program 200 comprises one or more modules having the necessary routines and data structures for performing specific tasks, and one or more engines configured to determine how the platform manages and manipulates data. In some embodiments, the application program 200 comprises one or more of a communication module 202, a database engine 204, an ROI calculation module 210, a user module 212, a patient module 214, and a display module 216.

In some embodiments, the communication module 202 is configured for receiving, processing, and transmitting a user command and/or one or more data streams. In such embodiments, the communication module 202 performs communication functions between various devices, including the user computing device 145, the administrator computing device 185, and a third-party computing device 195. In some embodiments, the communication module 202 is configured to allow one or more users of the system, including a third-party, to communicate with one another. In some embodiments, the communications module 202 is configured to maintain one or more communication sessions with one or more servers, the administrative computing device 185, and/or one or more third-party computing device(s) 195.

In some embodiments, a database engine 204 is configured to facilitate the storage, management, and retrieval of data to and from one or more storage mediums, such as the one or more internal databases described herein. In some embodiments, the database engine 204 is coupled to an external storage system. In some embodiments, the database engine 204 is configured to apply changes to one or more databases. In some embodiments, the database engine 204 comprises a search engine component for searching through thousands of data sources stored in different locations.

In some embodiments, the ROI calculation module 210 is configured to receive operational metrics to determine aspects of patient and business interactions to aid in the calculation of an ROI. These can include average time to appointment, no show information, and recalls. Average time to appointment may include calculating the average number of business days that a particular office location is open and determining the difference or delta between the date/time for a marketing lead to contact the office and the date/time when the marketing lead actually arrives at the office for an appointment. Various other information may be used including no-show information which is determine by calculating the date/time when an appointment is scheduled and then after a patient or marketing lead was scheduled to show up, using PMS data to determine if the appointment occurred as scheduled or if the patient or marketing lead failed to show up. Recalls can be determined by searching for appointments that are scheduled in the future that have specific codes (e.g., dental codes) that denote that a particular patient or marketing lead is returning in the future for a hygiene or other appointment.

In some embodiments, the user module 212 facilitates the creation of a user account for the application system. The user module 212 may allow the user to create a user profile which includes user information, user preferences, and user-associated information.

In some embodiments, the patient module 214 is in operable communication with the computing device to monitor various aspects of patient interactions with system and/or business. Interactions may include phone call interactions, form interactions, chat interactions, and scheduler interactions data can be used to create or otherwise be assigned as marketing lead data object(s). This patient data can be injected into one or more databases (e.g., a patient database). Similarly, marketing lead data objects can be injected into one or more databases (e.g., a marketing database). Information from these databases can then be subjected to matching logic which may include phone, email, name, appointment time, and/or other matching logic functions. Matching logic may be utilized and may be static in some instances, but in most instances, it will be frequently or constantly evolving in order to increase confidence intervals related to the data.

In some embodiments, the display module 216 is configured to display one or more graphic user interfaces, including, e.g., one or more user interfaces, one or more consumer interfaces, one or more video presenter interfaces, etc. In some embodiments, the display module 216 is configured to temporarily generate and display various pieces of information in response to one or more commands or operations. The various pieces of information or data generated and displayed may be transiently generated and displayed, and the displayed content in the display module 216 may be refreshed and replaced with different content upon the receipt of different commands or operations in some embodiments. In such embodiments, the various pieces of information generated and displayed in a display module 216 may not be persistently stored. The display module 216 provides alerts to the user device which can be viewed and acknowledged by the user.

In this disclosure, the various embodiments are described with reference to the flowchart illustrations and/or block diagrams of methods, apparatus (systems), and computer program products. Those skilled in the art would understand that each block of the flowchart illustrations and/or block diagrams, and combinations of blocks in the flowchart illustrations and/or block diagrams, can be implemented by computer readable program instructions. The computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions or acts specified in the flowchart and/or block diagram block or blocks. The computer readable program instructions can be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. The computer readable program instructions can be loaded onto a computer, other programmable data processing apparatus, or other device to cause a series of operational acts to be performed on the computer, other programmable apparatus, or other device to produce a computer implemented process, such that the instructions that execute on the computer, other programmable apparatus, or other device implement the functions or acts specified in the flowchart and/or block diagram block or blocks.

In this disclosure, the block diagrams in the Figures illustrate the architecture, functionality, and operation of possible implementations of systems, methods, and computer program products according to the various embodiments. Each block in the flowchart or block diagrams can represent a module, segment, or portion of instructions, which comprises one or more executable instructions for implementing the specified logical function(s). In some embodiments, the functions noted in the blocks can occur out of the order noted in the Figures. For example, two blocks shown in succession can, in fact, be executed concurrently or substantially concurrently, or the blocks can sometimes be executed in the reverse order, depending upon the functionality involved. In some embodiments, each block of the block diagrams and/or flowchart illustration, and combinations of blocks in the block diagrams and/or flowchart illustration, can be implemented by a special purpose hardware-based system that performs the specified functions or acts or carry out combinations of special purpose hardware and computer instructions.

In this disclosure, the subject matter has been described in the general context of computer-executable instructions of a computer program product running on a computer or computers, and those skilled in the art would recognize that this disclosure can be implemented in combination with other program modules. Generally, program modules include routines, programs, components, data structures, etc. that perform particular tasks and/or implement particular abstract data types. Those skilled in the art would appreciate that the computer-implemented methods disclosed herein can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated embodiments can be practiced in distributed computing environments where tasks are performed by remote processing devices that are linked through a communications network. Some embodiments of this disclosure can be practiced on a stand-alone computer. In a distributed computing environment, program modules can be located in both local and remote memory storage devices.

In this disclosure, the terms “component,” “system,” “platform,” “interface,” and the like, can refer to and/or include a computer-related entity or an entity related to an operational machine with one or more specific functionalities. The disclosed entities can be hardware, a combination of hardware and software, software, or software in execution. For example, a component can be a process running on a processor, a processor, an object, an executable, a thread of execution, a program, and/or a computer. By way of illustration, both an application running on a server and the server can be a component. One or more components can reside within a process and/or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As another example, a component can be an apparatus that provides specific functionality through electronic components without mechanical parts, wherein the electronic components can include a processor or other means to execute software or firmware that confers at least in part the functionality of the electronic components. In some embodiments, a component can emulate an electronic component via a virtual machine, e.g., within a cloud computing system.

The phrase “application” as is used herein means software other than the operating system, such as Word processors, database managers, Internet browsers and the like. Each application generally has its own user interface, which allows a user to interact with a particular program. The user interface for most operating systems and applications is a graphical user interface (GUI), which uses graphical screen elements, such as windows (which are used to separate the screen into distinct work areas), icons (which are small images that represent computer resources, such as files), pull-down menus (which give a user a list of options), scroll bars (which allow a user to move up and down a window) and buttons (which can be “pushed” with a click of a mouse). A wide variety of applications is known to those in the art.

The phrases “Application Program Interface” and API as are used herein mean a set of commands, functions and/or protocols that computer programmers can use when building software for a specific operating system. The API allows programmers to use predefined functions to interact with an operating system, instead of writing them from scratch. Common computer operating systems, including Windows, Unix, and the Mac OS, usually provide an API for programmers. An API is also used by hardware devices that run software programs. The API generally makes a programmer's job easier, and it also benefits the end user since it generally ensures that all programs using the same API will have a similar user interface.

The phrase “central processing unit” as is used herein means a computer hardware component that executes individual commands of a computer software program. It reads program instructions from a main or secondary memory, and then executes the instructions one at a time until the program ends. During execution, the program may display information to an output device such as a monitor.

The term “execute” as is used herein in connection with a computer, console, server system or the like means to run, use, operate or carry out an instruction, code, software, program and/or the like.

In this disclosure, the descriptions of the various embodiments have been presented for purposes of illustration and are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein. Thus, the appended claims should be construed broadly, to include other variants and embodiments, which may be made by those skilled in the art.

What is claimed is:

1. A system for providing a patient management platform for a dental practice, the system comprising:
at least one user computing device in operable connection with a user network;
an application server in operable communication with the user network, the application server configured to host an application program for providing a patient management platform, the application program having a user interface module for providing access to the application program via the at least one user computing device;
a ROI calculation module to calculate an ROI for a dental practice using a plurality of patient profile information and a plurality of marketing information; and
a plurality of matching logic to generate operation metric calculations.

2. The system of claim 1, further comprising a patient module to monitor a plurality of patient interactions.

3. The system of claim 2, wherein the plurality of patient interactions includes a phone call interaction.

4. The system of claim 2, wherein the plurality of patient interactions includes a form interaction.

5. The system of claim 2, wherein the plurality of patient interactions includes a chat interaction.

6. The system of claim 2, wherein the plurality of patient interactions includes a scheduler interaction.

7. The system of claim 2, wherein each of the plurality of patient interactions are assigned as a marketing lead data object.

8. An integrated marketing and patient management platform system for a dental practice, comprising:
a server communicatively coupled to a network and including a processor that performs the steps of:
creating and storing a patient profile for a patient in non-transitory, computer readable memory;
creating and storing marketing information in non-transitory, computer readable memory;
accessing patient profile information from the patient profile and marketing information; and
performing one or more calculations using the patient profile information and marketing information to determine ROI of a patient.

9. The system of claim 8, further comprising matching logic to receive a plurality of patient information, the patient information including a phone number, an email, a name, and an appointment time.

10. The system of claim 9, wherein the matching logic is utilized to generate operation metric calculations.

11. The system of claim 8, further comprising a patient module to monitor a plurality of patient interactions.

12. The system of claim 8, wherein the plurality of patient interactions includes a phone call interaction.

13. The system of claim 8, wherein the plurality of patient interactions includes a form interaction.

14. The system of claim 8, wherein the plurality of patient interactions includes a chat interaction.

15. The system of claim 8, wherein the plurality of patient interactions includes a scheduler interaction.

16. The system of claim 8, wherein each of the plurality of patient interactions are assigned as a marketing lead data object.
